(19) United States
(12) Patent Application Publication
(10) Pub. No.: US 2004/0225531 A1
(43) Pub. Date: Nov. 11, 2004

(54) Computerized system and method for automated correlation of mammography report with pathology specimen result

(76) Inventors: Laura Kathleen Serrano, Lenexa, KS (US); John Lee Moore III, Independence, MO (US); Judith Ann Zakutny, Olathe, KS (US)

Correspondence Address: SHOOK, HARDY & BACON LLP, 2555 GRAND BLVD, KANSAS CITY,, MO, 64108, US

(21) Appl. No.: 10/431,548

(22) Filed: May 7, 2003

(51) Int. Cl. G06F 17/60; A61B 6/04

(52) U.S. Cl. 705/2; 378/37

(57) ABSTRACT

A computerized system and method for correlating pathology specimen results for breast tissue with mammography reports. Upon determining that one or more patients have both pathology results for breast tissue and one or more mammography reports, the method identifies the patients having both. The system includes a pathology entry component, a mammography entry component, a correlation component and a user component.

### CROSS-REFERENCE TO RELATED APPLICATIONS

None.

### STATEMENT REGARDING FEDERALLY SPONSORED RESEARCH OR DEVELOPMENT

None.

### FIELD OF THE INVENTION

The present invention relates generally to the field of computer software. More particularly, the invention relates to a computerized system and method for correlating pathology specimen results for breast tissue with mammography reports.

### BACKGROUND OF THE INVENTION

Mammography is an important tool in detecting breast cancer. While a mammogram alone cannot be used to diagnose a patient with breast cancer, it can indicate whether an abnormality in the breast is present.

Mammography is highly regulated by the Food and Drug Administration's Mammography Quality Standards Act (“MQSA”). According to MQSA, radiologists should make an assessment of the findings in one of the following categories: negative, benign, probably benign, suspicious, highly suggestive of malignancy or incomplete. Many radiologists use the BI-RADS system developed by the American College of Radiology (ACR). According to the BI-RADS system, radiologists issue assessment gradings of 0-5 for each patient mammogram. A grading of “0” indicates additional imaging is needed. A grading of “1,” “2” or “3” is usually considered negative for breast cancer and gradings of “4” and “5” are considered positive for breast cancer. Along with grading a mammogram report, the radiologist may recommend that a patient have a breast biopsy, aspiration, surgical procedure or other interventional tissue diagnosis procedure if there is an abnormality to determine whether or not the patient actually has breast cancer.

The MQSA requires that each facility performing mammograms perform a medical outcomes audit of radiologist(s) mammography assessments. The general requirements of MQSA require each mammography facility to establish a system to collect and review surgical and/or pathology results of a breast tissue for all patients imaged at that facility. This must be done for both individual physicians and collectively for all physicians who interpret mammograms at the facility. The MQSA does not require that each facility perform specific calculations. However, it is recommended that the facility determine the percentage of exams interpreted as positive that have a positive pathology report and the percentage of exams interpreted as positive that do not have a positive pathology report.

Currently, a radiology facility performing mammograms must manually follow-up on all positive mammography reports and call the testing laboratory to obtain any pathology results for a patient imaged at their facility. Often, the facility receives results from pathology laboratories containing all pathology reports for all patients. The facility must search through these pathology results to find the pathology results for any patients imaged at their facility. The pathology results must then be manually compared with the mammography reports. This current process is time-consuming and prone to human error.

### SUMMARY OF THE INVENTION

The present invention overcomes the drawbacks of the current manual process. In one aspect of the present invention, a method in a computing environment for correlating mammography reports with pathology results for one or more patients is provided. The method comprises determining whether one or more patients have both pathology results for breast tissue and one or more mammography reports and, if so, identifying patients having both.

Another aspect of the present invention is a computerized method for correlating mammography reports with pathology results for one or more patients. The method comprises: receiving pathology results for one or more patients; determining whether one or more patients have both pathology results for breast tissue and one or more mammography reports and if so, placing the pathology results in a correlation queue.

In yet another aspect of the present invention. A computer system for correlating mammography reports with pathology results for one or more patients is provided. The system comprises a determination component for determining whether one or more patients have both pathology results for breast tissue and one or more mammography reports and an identification component for identifying one or more patients having both pathology results for breast tissue and one or more mammography reports.

In a further aspect of the invention, a computer system for correlating mammography reports with pathology results for one or more patients is provided. The system comprises: a pathology result entry component for input of pathology results for one or more patients; a mammography report entry component for input of one or more mammography reports for one or more patients; a determination component for determining whether one or more patients have both pathology results for breast tissue and one or more mammography reports; an identification component for identifying one or more patients having both pathology results for breast tissue and one or more mammography reports; and a user component for allowing one or more users to access the pathology results and one or more mammography reports for one or more patients having both.

Still a further aspect of the invention provides a computer system for correlating mammography reports with pathology results for one or more patients. The system comprises a means for determining whether one or more patients have both pathology results for breast tissue and one or more mammography reports and a means for identifying one or more patients having both pathology results for breast tissue and one or more mammography reports.

Still a further aspect of the invention provides one or more computer-readable media having a data structure stored thereon, the data structure comprises a first field containing data indicative of pathology results for one or more patients; a second field containing data indicative of one or more mammography reports for one or more patients; and a third field containing data indicative of one or more patients having both pathology results and one or more mammography reports.

In yet another aspect of the invention, a method in a computing environment is provided. The method comprises: determining whether one or more patients have both pathology results for breast tissue and one or more mammography reports and, if so, correlating the mammography reports with the pathology results for a patient having both.

### BRIEF DESCRIPTION OF THE DRAWING

The present invention is described in detail below with reference to the attached drawing figures, wherein:

FIG. 1 is a block diagram of a computing system including a correlation component in accordance with an embodiment of the present invention;

FIG. 2 is a flowchart representative of a computer program for correlating pathology results with mammography reports in accordance with an embodiment of the present invention;

FIG. 3 is a screen shot illustrating an exemplary implementation of the correlation of pathology results and mammography reports; and

FIG. 4 is a block diagram of a data structure containing pathology result information, mammography information and data identifying one or more patients having both in accordance with an embodiment of the present invention.

### DETAILED DESCRIPTION OF THE INVENTION

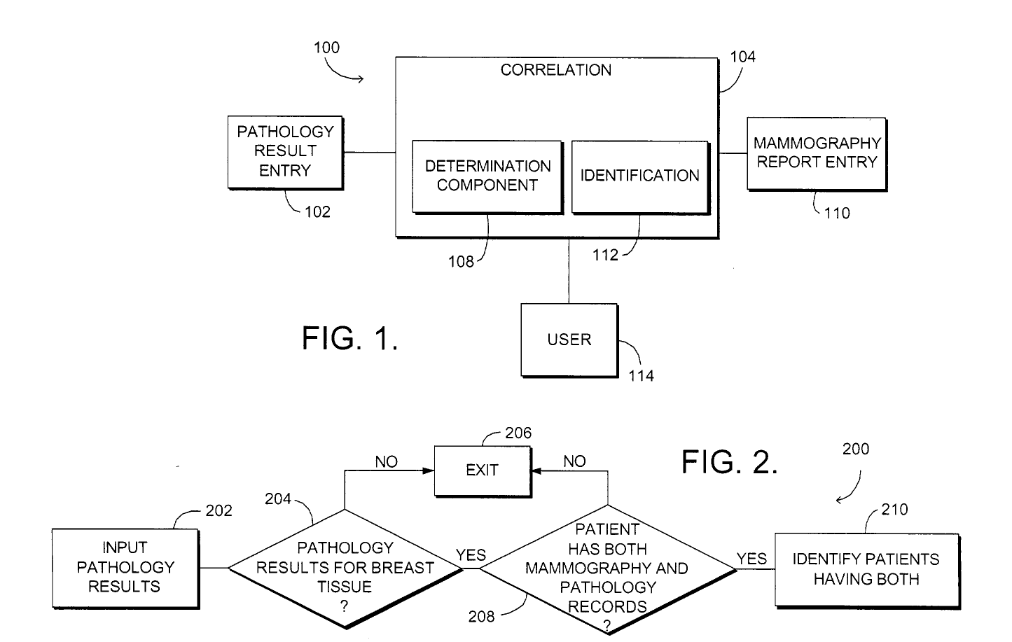

The present invention is directed to a computerized system and method for correlating pathology results with mammography reports issued by radiology. FIG. 1 depicts an exemplary computer system 100 for correlating pathology results with mammography reports. The system 100 includes a pathology result entry component 102 and a mammography report entry component 110 coupled with a data correlation component 104. The correlation component 104 includes two subcomponents. Subcomponent 108 determines whether one or more patients have both pathology results for breast tissue and one or more mammography reports. Subcomponent 112 identifies patients who have both pathology and mammography reports. The system 100 also includes user component 114 for accessing the mammography reports and pathology results for one or more patients having both.

Those skilled in the art will appreciate that the present invention contemplates the presence of additional components and/or subcomponents of the computer system 100, and the components and/or subcomponents may be combined with one another and/or separated into new components and subcomponents. For example, the mammography report entry component 110, the user component 114, and correlation component 104 may be combined with one another. The combined components receive pathology results and determine whether one or more patients have both pathology results and mammography reports. In this example, the system identifies one or more patients having both by placing the pathology results in a correlation queue for review by qualified personnel, such as the radiological technologist.

The present invention may be implemented in a variety of computing system environments. For example, each of the components and subcomponents of the computer system 100 may be embodied in an application program running on one or more personal computers (PCs). This computing system environment is only one example of a suitable computing environment and is not intended to suggest any limitation as to the scope of use or functionality of the invention. The invention may also be implemented with numerous other general purpose or special purpose computing system environments or configurations. Examples of other well-known computing systems, environments, and/or configurations that may be suitable for use with the invention include, but are not limited to, server computers, hand-held or laptop devices, multiprocessor systems, microprocessor-based systems, programmable consumer electronics, network PCs, minicomputers, mainframe computers, distributed computing environments that include any of the above systems or devices, and the like.

The invention may be described in the general context of computer-executable instructions, such as program modules. Generally, program modules include routines, programs, objects, components, segments, schemas, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

Computers typically include a variety of computer-readable media. Computer-readable media includes any media that can be accessed by a computer and includes both volatile and nonvolatile media, removable and nonremovable media. By way of example, and not limitation, computer-readable media may comprise computer storage media and communications media. Computer storage media includes both volatile and nonvolatile, removable and nonremovable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD), holographic or other optical storage, magnetic cassettes, magnetic tape, magnetic disk storage or other magnetic storage devices, or any other medium which can be used to store the desired information and which can be accessed by a computer.

Communications media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communications media includes wired media such as a wired network or direct wired connection, and wireless media such as acoustic, RF, infrared, spread spectrum and other wireless media. Communications media are commonly used to upload and download information in a network environment, such as the Internet. Combinations of any of the above should also be included within the scope of computer-readable media.

The computer may operate in a networked environment using logical connections to one or more remote computers, such as a personal computer, a server, a router, a network PC, a peer device or other common network node, and typically includes many or all of the elements described above. The logical connections may include connections to a local area network (LAN), a wide area network (WAN) and/or other networks. Such networking environments are commonplace in offices, enterprise-wide computer networks, intranets and the Internet.

Computer storage mechanisms and associated media provide storage of computer-readable instructions, data structures, program modules and other data for the computer. A user may enter commands and information into the computer through input devices such as a keyboard and pointing device, commonly referred to as a mouse, trackball or touch pad. Other input devices may include a microphone, touchscreen, camera, joystick, game pad, scanner, or the like. In addition to a monitor or other type of display device, computers may also include other peripheral output devices such as speakers and printers, which may be connected through an output peripheral interface.

Although many other internal components of computers have not been discussed herein, those of ordinary skill in the art will appreciate that such components and their interconnection are well known. Accordingly, additional details concerning the internal construction of computers need not be disclosed in connection with the present invention.

There are commercially available software applications for processing mammography reports, such as the RadNet® application marketed by Cerner Corporation of Kansas City, Mo. These systems automate some steps of the medical outcomes audit required by MQSA. Specifically, the RadNet® application identifies positive mammography reports that require follow up with the patient and provides a method to follow up with the patient. Mammography reports may also be contained and processed by other systems. There also are commercially available software systems for processing and storing pathology results, such as the Cerner Millennium™ repository, the Cerner Millennium™ system, and the HNA Classic™ system of Cerner Corporation. Pathology results may also be stored in other systems. The pathology results stored in these systems may be accessed by the system of the present invention.

Referring next to FIG. 2, a flow diagram 200 is provided which illustrates an embodiment for correlating pathology results with mammography reports in accordance with the present invention. After pathology results are input at block 202, it is determined at decision block 204 whether or not the pathology results are for breast tissue. If not, no further action is taken and the system is exited at block 206. In an alternative embodiment, only pathology results for breast tissue are input and decision block 204 is bypassed.

However, if it is determined at decision block 204 that the pathology results for the patient are for breast tissue, then it is determined at decision block 208 whether one or more patients have both pathology results and one or more mammography reports. If not, no further action is taken and the system again is exited at block 206. If the determination at decision block, 208 is affirmative, one or more patients having both are identified at block 210. One of skill in the art will understand that identifying patients having both mammography reports and pathology results may be done in any number of ways including, but not limited to, updating the system to reflect one or more patients having both pathology results and mammography reports, making a list of patients who have both, and placing the pathology results for a patient having both in a correlation queue for review by qualified personnel.

By way of example, pathology results for a fictitious patient, Naomi Danner, are input at block 202. At block 204, the system determines the pathology results are for breast tissue. At block 208, it is determined that the patient, Naomi Danner, has both pathology results and a mammography report. Naomi Danner is identified as having both a mammography report and pathology results by placing her pathology results in a correlation queue to be reviewed by qualified radiological personnel.

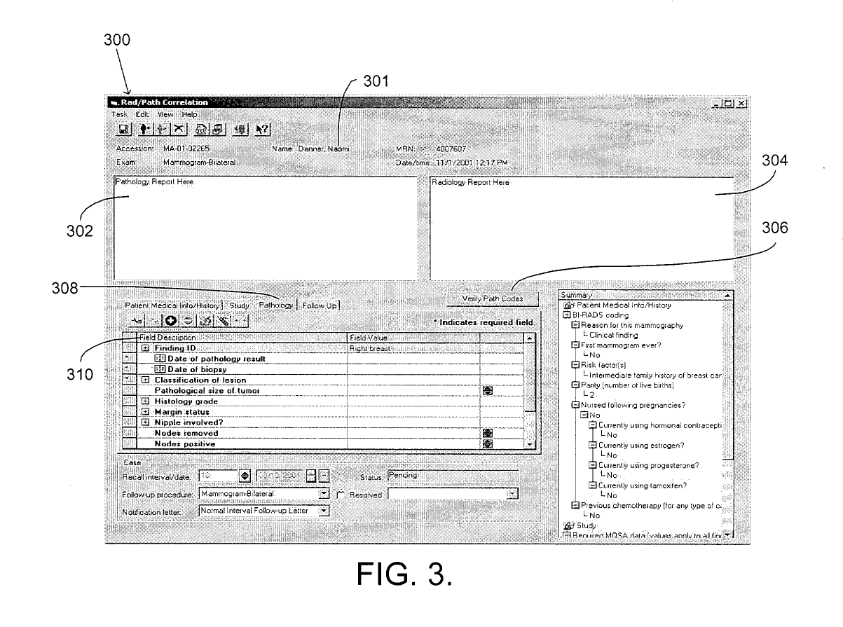

Referring now to FIG. 3, a screen shot of an exemplary implementation of the pathology correlation window 300 is illustrated. The correlation window is accessed by selecting the desired pending patient pathology results listed in the correlation queue. The pathology correlation window displays the pathology result text 302 and mammography report text 304 for a patient identified at display 301. If there are multiple mammogram reports for one patient, the user will determine to which record the pathology results correspond. From this screen, one or more users, such as a radiologist, may code the pathology report in a box 308 to be utilized to compare the radiologist's assessment gradings with the actual pathology/biopsy results. The coding may be done in accordance with ACR standards and may include various information regarding pathology results including, but not limited to, malignant or benign diagnoses, histologic grade and nodal involvement. In the alternative, the user may verify pre-coded pathology reports that have already been coded by the pathologist by clicking a button 306. Once the pathology results have been coded or verified, the system will remove the case from pathology correlation queue.

Continuing the above example, a user selects Naomi Danner's pathology results from the correlation queue. The system displays both the pathology result text and the mammography report text for Naomi Danner in the pathology correlation window 300. In this example, the pathology results text was not pre-coded, so the radiologist who interpreted her mammography results will properly code the pathology results. Naomi had a grading of “5” on her mammography report indicating a positive result and her pathology results were also positive for breast cancer. The interpreting radiologist codes the pathology results to reflect the malignancy. Then the system collects and stores the mammography assessment and coded pathology results for the facility in accordance with the MQSA.

Statistical audits, in accordance with MQSA, may be done to compare the radiology assessment with the coded pathology results. For example, statistical analyses may be done to calculate the percentage of exams interpreted by a radiologist as positive that have a positive pathology report and to calculate the percentage of exams interpreted by a radiologist as positive that do not have a positive pathology report. In the above example, Naomi Danner's mammography report and coded pathology results will be utilized in the statistical calculation of the percentage of exams that were interpreted as positive by a radiologist and have positive pathology results.

Statistical audits, in accordance with MQSA, may be done to compare the radiology assessment with the coded pathology results. For example, statistical analyses may be done to calculate the percentage of exams interpreted by a radiologist as positive that have a positive pathology report and to calculate the percentage of exams interpreted by a radiologist as positive that do not have a positive pathology report. In the example displayed in FIG. 3, Naomi Danner's mammography report, and is correlation to the coded pathology results, will be utilized in the statistical calculation of positive predictive value for the individual radiologist and the aggregate of all radiologists at the facility.

Positive predictive value (PPV) is defined as the percentage of all positive mammographic examinations which have a tissue diagnosis of cancer within one year of the examination date. As known in the art, the formula for calculating positive predictive value is as follows:

PPV=True Positive/(True Positive+False Positive)

Thus, if an examination is interpreted as positive and the pathology result is positive, then the classification is True Positive. In another alternative, if an examination is interpreted as positive but a negative pathology result is returned, the classification is False Positive. In yet another alternative, if an examination is interpreted as negative by a Radiologist but a positive pathology result is returned, the classification is False Negative. If an examination is interpreted as negative, and later a negative pathology result is returned, then the classification is True Negative.

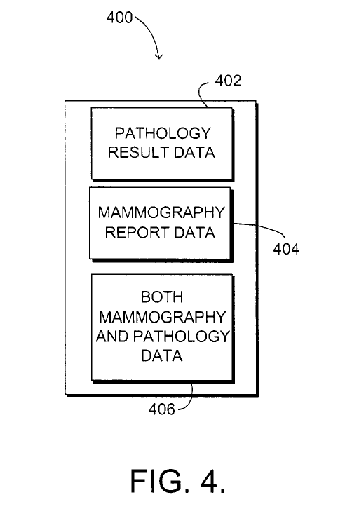

With reference to FIG. 4, an exemplary data structure 400 containing correlation data in accordance with an embodiment of the present invention is shown. The data structure 400 includes a field 402 that contains data indicative of pathology results of breast tissue for one or more patients and a field 404 that contains data indicative of mammography report data for one or more patients. The data structure 400 also includes a field 406 identifying one or more patients who have both pathology results for breast tissue and one or more mammography reports. As is well known in the art, the data structure 400 may be stored on one or more computer readable media, and the data structure 400 may also contain many additional, optional fields.

One of skill in the art will appreciate the present invention provides a system and method for correlating pathology specimen results for breast tissue with mammography reports. The present invention determines whether one or more patients have both pathology results for breast tissue and one or more mammography reports, and if so, identifies the patients having both. The system includes a pathology entry component, a mammography entry component, a correlation component that includes a determination component and an identification component, and a user component.

Alternative embodiments and implementations of the present invention will become apparent to those skilled in the art to which it pertains upon review of the specification, including the drawing figures. Accordingly, the scope of the present invention is defined by the appended claims rather than the foregoing description.

What the invention claimed is:

1. A method in a computing environment for correlating mammography reports with pathology results for one or more patients, the method comprising:
determining whether one or more patients have both pathology results for breast tissue and one or more mammography reports; and
if so, identifying patients having both.

2. The method of claim 1, wherein identification is accomplished by updating the system to reflect patients having both pathology results and one or more mammography reports.

3. The method of claim 1, wherein identification is accomplished by listing patients having both pathology and mammography reports.

4. The method of claim 1, further comprising:
providing access to pathology results and mammography reports for one or more patients having both.

5. The method of claim 1, wherein identification is accomplished by placing the pathology results in a correlation queue for review.

6. The method of claim 5, further comprising:
displaying the correlation queue.

7. The method of claim 6, further comprising:
providing access to pathology results and mammography reports for one or more patients having both.

8. The method of claim 7, wherein the pathology results may be coded by the user to be utilized for statistical analysis.

9. The method of claim 7, wherein the pathology results are pre-coded.

10. The method of claim 9, wherein the pre-coded pathology results may be verified a user.

11. One or more computer-readable media having computer-executable instructions for performing the method for correlating mammography reports with pathology results for one or more patients, the method comprising:
determining whether one or more patients have both pathology results for breast tissue and one or more mammography reports; and
if so, identifying patients having both.

12. A computerized method for correlating mammography reports with pathology results for one or more patients, the method comprising:
receiving pathology results for one or more patients;
determining whether one or more patients have both pathology results for breast tissue and one or more mammography reports; and
if so, placing the pathology results in a correlation queue.

13. The method of claim 12, further comprising:
displaying the correlation queue.

14. The method of claim 13, further comprising:
providing access to pathology results and mammography reports for one or more patients having both.

15. The method of claim 14, wherein the pathology results may be coded by one or more users to be utilized for statistical analysis.

16. The method of claim 14, wherein the pathology results are pre-coded.

17. The method of claim 16, wherein the pre-coded pathology results may be verified by a user.

18. One or more computer-readable media having computer-executable instructions for performing the method recited in claim 12.

19. A computer system for correlating mammography reports with pathology results for one or more patients, the system comprising:
a determination component for determining whether one or more patients have both pathology results for breast tissue and one or more mammography reports; and
an identification component for identifying one or more patients having both pathology results for breast tissue and one or more mammography reports.

20. A computer system for correlating mammography reports with pathology results for one or more patients, the system comprising:
a pathology result entry component for input of pathology results for one or more patients;
a mammography report entry component for input of one or more mammography reports for one or more patients;
a determination component for determining whether one or more patients have both pathology results for breast tissue and one or more mammography reports;
an identification component for identifying one or more patients having both pathology results for breast tissue and one or more mammography reports; and
a user component for allowing one or more users to access the pathology results and one or more mammography reports for one or more patients having both.

21. A computer system for correlating mammography reports with pathology results for one or more patients, the system comprising:
a means for determining whether one or more patients have both pathology results for breast tissue and one or more mammography reports; and
a means for identifying one or more patients having both pathology results for breast tissue and one or more mammography reports.

22. One or more computer-readable media having a data structure stored thereon, the data structure comprising:
a first field containing data indicative of pathology results for one or more patients;
a second field containing data indicative of one or more mammography reports for one or more patients; and
a third field containing data indicative of one or more patients having both pathology results and one or more mammography reports.

23. The one or more computer-readable media of claim 22, wherein the data structure further comprises a fourth data field containing data indicative of identifying one or more patients having both pathology results and one or more mammography reports.

24. A method in a computing environment, the method comprising:
determining whether one or more patients have both pathology results for breast tissue and one or more mammography reports; and
if so, correlating the mammography reports with the pathology results for a patient having both.
